(19) United States
(12) Patent Application Publication
(10) Pub. No.: US 2019/0271771 A1
(43) Pub. Date: Sep. 5, 2019

(54) SEGMENTED COMMON ANATOMICAL STRUCTURE BASED NAVIGATION IN ULTRASOUND IMAGING

(71) Applicant: BK Medical Holding Company, Inc., Peabody, MA (US)

(72) Inventors: David Lieblich, Worcester, MA (US); Li Zhaolin, Lynn, MA (US)

(73) Assignee: BK Medical Holding Company, Inc., Peabody, MA (US)

(21) Appl. No.: 16/302,193

(22) PCT Filed: May 16, 2016
(86) PCT No.: PCT/US2016/032647

§ 371 Date: Nov. 16, 2018

(51) Int. Cl. G01S 7/52; G06K 9/32; A61B 8/08

(52) U.S. Cl. CPC G01S 7/52036; G01S 7/5206; G01S 7/52074; G06K 9/3208; G16H 30/40; G06T 2207/10132; G06T 2207/30004; G06T 2207/30244; A61B 8/5223

(57) ABSTRACT

A method includes obtaining a real-time 2-D B-mode image of anatomy of interest in a region of interest. The real-time 2-D B-mode image is generated with ultrasound echoes received by transducer elements (106) of a transducer array (104). The method further includes segmenting one or more anatomical features from the real-time 2-D B-mode image, obtaining 2-D slices of anatomically segmented 3-D navigation image data for the same region of interest, and matching the real-time 2-D B-mode image to at least a sub-set of the 2-D slices based on the segmented anatomical features. The method further includes identifying a 2-D slice of the anatomically segmented 3-D navigation image data that matches the real-time 2-D B-mode image based on the matching, and identifying at least one of a location and an orientation of the transducer array relative to the anatomy based on the match.

### TECHNICAL FIELD

The following generally relates to image guided navigation and more particularly to segmented common anatomical structure based navigation, and is described with particular application to ultrasound imaging, but is also amenable to other imaging modalities.

### BACKGROUND

An ultrasound imaging system has included a transducer array that transmits an ultrasound beam into an examination field of view. As the beam traverses structure (e.g., in an object or subject, etc.) in the field of view, sub-portions of the beam are attenuated, scattered, and/or reflected off the structure, with some of the reflections (echoes) traversing back towards the transducer array. The transducer array receives and processes the echoes, and generates one or more images of the subject or object and/or instrument. The resulting ultrasound images have been used to navigate or guide procedures in real-time (i.e., using presently generated images from presently acquired echoes). Navigation, generally, can be delineated into navigation based on an external navigation system (e.g., electromagnetic based navigation system, etc.) and navigation not based on an external navigation system (e.g., free hand).

Navigation based on an external navigation system adds navigation components (e.g., a sensor, etc.), which increases overall complexity and cost of the system. With non-external navigation based systems, one approach is to rely on extraction of positioning information from the real-time data alone. This may include using a finite distance correlation of speckle imposed by a beam width, in the elevation direction, and its variation with depth, to obtain an estimate of transducer displacement between two image planes. Alternatively, this may include using a uniqueness of imaged anatomy within a 2-D image plane(s) to determine location within the target anatomy. This relies on an accurate 3-D model of the imaged anatomy, and sufficient uniqueness of the 2-D planar intersections of that anatomy to provide 3-D positioning of sufficient accuracy and timeliness for the required navigation.

### SUMMARY

Aspects of the application address the above matters, and others. In one aspect, a method includes obtaining a real-time 2-D B-mode image of anatomy of interest in a region of interest. The real-time 2-D B-mode image is generated with ultrasound echoes received by transducer elements of a transducer array. The method further includes segmenting one or more anatomical features from the real-time 2-D B-mode image, obtaining 2-D slices of anatomically segmented 3-D navigation image data for the same region of interest, and matching the real-time 2-D B-mode image to at least a sub-set of the 2-D slices based on the segmented anatomical features. The method further includes identifying a 2-D slice of the anatomically segmented 3-D navigation image data that matches the real-time 2-D B-mode image based on the matching, and identifying at least one of a location and an orientation of the transducer array relative to the anatomy based on the match.

In another aspect, an apparatus includes a navigation processor configured to segment at least one anatomical organ of interest in a real-time 2-D ultrasound image and match the real-time 2-D ultrasound image to a 2-D slice of an anatomical segmented 3-D volume of image of interest based on a common segmented anatomy in the real-time 2-D ultrasound image and the anatomical segmented 3-D volume of image of interest.

In another aspect, a non-transitory computer readable medium is encoded with computer executable instructions, which, when executed by a computer processor, causes the processor to: segment one or more structure in a real-time 2-D ultrasound image, match a contour of at least one of the segmented structures of the real-time 2-D ultrasound image with one or more contours of segmented anatomy in one or more planar cuts of 3-D image data including a planar cut corresponding to the real-time 2-D ultrasound image, determine a location and an orientation of a transducer array to obtain the real-time 2-D ultrasound image relative to the 3-D image data based on the match, and display the 3-D D image data with the real-time 2-D ultrasound image superimposed thereover at the determined location and orientation.

Those skilled in the art will recognize still other aspects of the present application upon reading and understanding the attached description.

### BRIEF DESCRIPTION OF THE DRAWINGS

The application is illustrated by way of example and not limitation in the figures of the accompanying drawings, in which like references indicate similar elements and in which:

FIG. 1 schematically an example ultrasound imaging system with a navigation processor;

FIG. 2 schematically an example of the navigation processor;

FIG. 3 depicts an example of a real-time 2-D ultrasound image with at least sub-portions of segmented anatomical structures;

FIG. 4 depicts an example of an anatomical structure segmented in 3-D navigation reference image data;

FIG. 5 depicts an example of a planar cut through the 3-D navigation reference image data, including the segmented anatomical structure;

FIG. 6 depicts an example of another planar cut through the 3-D navigation reference image data, including the segmented anatomical structure;

FIG. 7 depicts an example of yet another planar cut through the 3-D navigation reference image data, including the segmented anatomical structure; and

FIG. 8 illustrates an example method in accordance with an embodiment herein.

### DETAILED DESCRIPTION

The following generally describes an approach for real-time anatomically-based navigation for a procedure. In one instance, this approach includes segmenting predetermined anatomy in a real-time 2-D ultrasound image, optionally using knowledge of a relative location and boundary of segmented anatomic structures in previously generated 3-D reference segmentation image data, where the real-time 2-D ultrasound image intersects a scan plane(s) in 3-D reference navigation image data having previously segmented 3-D anatomical structures. The real-time 2-D ultrasound image is matched to a planar cut in the 3-D reference navigation image data based on the segmented anatomy common in both data sets, which maps a location of the real-time 2-D ultrasound image to the 3-D reference navigation image data and, thereby, a current location and orientation of an ultrasound probe for the procedure.

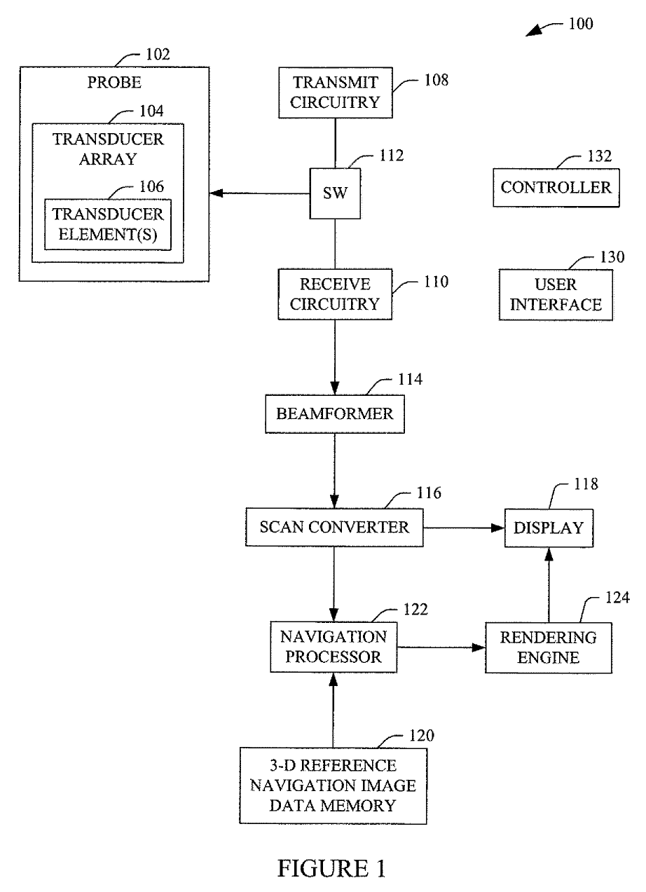

Initially referring to FIG. 1, an example imaging system such as an ultrasound (US) imaging system 100 is schematically illustrated.

The ultrasound imaging system 100 includes a probe 102 housing a transducer array 104 having at least one transducer element 106. The at least one transducer element 106 is configured to convert electrical signals to an ultrasound pressured field and vice versa respectively to transmit ultrasound signals into a field of view and receive echo signals, generated in response to interaction with structure in the field of view, from the field of view. The transducer array 104 can be linear, curved (e.g., concave, convex, etc.), circular, etc., fully populated or sparse, etc.

Transmit circuitry 108 generates a set of pulses (or a pulsed signal) that are conveyed, via hardwire (e.g., through a cable) and/or wirelessly, to the transducer array 104. The set of pulses excites a set (i.e., a sub-set or all) of the at least one transducer element 106 to transmit ultrasound signals. Receive circuitry 110 receives a set of echoes (or echo signals) generated in response to a transmitted ultrasound signal interacting with structure in the field of view. A switch (SW) 112 controls whether the transmit circuitry 108 or the receive circuitry 110 is in electrical communication with the at least one transducer element 106 to transmit ultrasound signals or receive echoes.

A beamformer 114 processes the received echoes by applying time delays to echoes, weighting echoes, summing delayed and weighted echoes, and/or otherwise beamforming received echoes, creating beamformed data. In B-mode imaging, the beamformer 114 produces a sequence of focused, coherent echo samples along focused scanlines of a scanplane. The beamformer 114 may also process the scanlines to lower speckle and/or improve specular reflector delineation via spatial compounding, and/or perform other processing such as FIR filtering, IIR filtering, edge enhancement, etc.

A scan converter 116 scan converts the output of the beamformer 114 to generate data for display, e.g., by converting the data to the coordinate system of a display 118. The scan converter 116 can be configured to employ analog and/or digital scan converting techniques. The display 118 can be a light emitting diode (LED), liquid crystal display (LCD), and/or type of display, which is part of the ultrasound imaging system 100 or in electrical communication therewith via a cable.

A 3-D reference navigation image data memory 120 includes previous generated and segmented 3-D reference navigation image data having one or more segmented 3-D anatomical structures. In general, the 3-D reference navigation image data includes a 3-D volume of the anatomy in which the tissue or interest, or target tissue, is located. In one instance, 3-D reference navigation image data corresponds to a scan performed prior to the examination procedure and can be generated by a same modality as the imaging system 100 (with the same or different settings) and/or a different modality (e.g., magnetic resonance imaging (MRI), computed tomography (CT).

A non-limiting example of generating a 3-D volume from 2-D images acquired using a freehand probe rotation or translation is described in patent application serial number PCT/US2016/32639, filed May 16, 2016, entitled “3-D US VOLUME FROM 2-D IMAGES FROM FREEHAND ROTATION AND/OR TRANSLATION OF ULTRASOUND PROBE,” the entirety of which is incorporated herein by reference. Other approaches are also contemplated herein.

A navigation processor 122 maps a real-time 2-D ultrasound image generated by the imaging system 100 to a corresponding image plane in the 3-D reference navigation image data. As described in greater detail below, in one instance this is achieved by matching at least a sub-portion (e.g., a contour) of at least one segmented structure in the real-time 2-D ultrasound image with at least a sub-portion of at least one segmented 3-D anatomical structure of the 3-D reference navigation image data. As utilized herein, a real-time 2-D ultrasound image refers to a currently or presently generated image, generated from echoes currently or presently acquired with the transducer array 104.

A rendering engine 124 combines the real-time 2-D ultrasound image with the 3-D reference navigation image data at the matched image plane and visually presents the combined image data via the display 118 and/or other display. The resulting combination identifies the location and/or orientation of the ultrasound transducer 104 relative to the anatomy in the 3-D reference navigation image data. The display 118 is configured to display images, including the real-time 2-D ultrasound image, planar slices through the 3-D reference navigation image data, the 3-D reference navigation image data, the combined image data, and/or other data.

The anatomical navigation approach described herein, where anatomy is used to position the ultrasound probe within an volume of interest, may provide competitive advantage for a fusion product and/or an ultrasound-only product where the ultrasound is positioned in real-time. This can apply to any ultrasound probe, if a prior 3-D image data set, either from another modality or from the ultrasound probe itself e.g., without an expensive 3-D positioning system, is obtained and segmented.

A user interface (UI) 130 includes an input device(s) (e.g., a physical button, a touch screen, etc.) and/or an output device(s) (e.g., a touch screen, a display, etc.), which allow for interaction between a user and the ultrasound imaging system 100. A controller 132 controls one or more of the components 104-130 of the ultrasound imaging system 100. Such control includes controlling one or more of these components to perform the functions described herein and/or other functions.

In the illustrated example, at least one of the components of the system 100 (e.g., the navigation processor 122) can be implemented via one or more computer processors (e.g., a microprocessor, a control processing unit, a controller, etc.) executing one or more computer readable instructions encoded or embodied on computer readable storage medium (which excludes transitory medium), such as physical computer memory, which causes the one or more computer processors to carry out the various acts and/or other functions and/or acts. Additionally or alternatively, the one or more computer processors can execute instructions carried by transitory medium such as a signal or carrier wave.

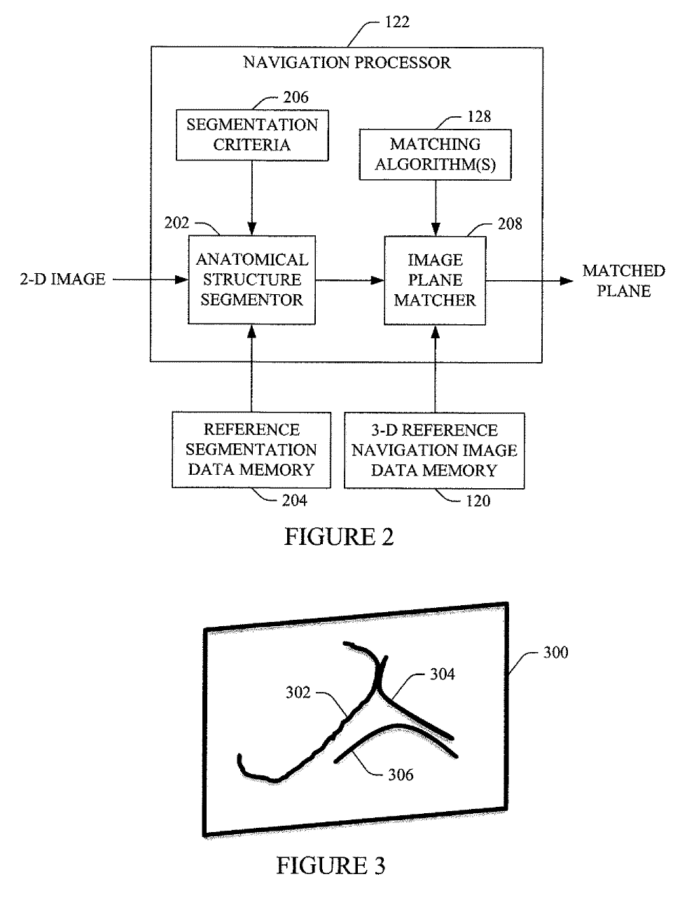

FIG. 2 schematically illustrates an example of the navigation processor 122.

The navigation processor 122 includes an anatomical structure segmentor 202 configured to segment at least one predetermined anatomical structure from the real-time 2-D ultrasound image using automatic and/or semi-automatic segmentation algorithms In the illustrated embodiment, the anatomical structure segmentor 202 utilizes knowledge of relative locations and boundaries of segmented anatomic structures in prior 3-D reference segmentation image data and/or an anatomical model of the region of interest stored from reference segmentation data memory 204 to segment the at least one anatomical structure. In a variation, the navigation processor 122 does not use this information. In this instance, this information and/or the memory 204 can be omitted.

The anatomical structure segmentor 202 additionally or alternatively segments based on predetermined segmentation criteria from criteria memory 206. An example of such criteria includes a number of anatomical structures to segment, an identity of the anatomical structures to segment, etc. In one instance, this information can be dynamically determined to achieve a predetermined tradeoff between sufficient positioning accuracy, in any given plane, and speed of update. Such an adjustment may account for a uniqueness of positioning afforded by different combinations of anatomical structures, based upon prior quantitative results, as well as considerations of data rate and speed of motion. These considerations may allow skipping of positioning on some planes.

An image plane matcher 208 is configured to match one or more of the anatomical structure segmented in the real-time 2-D ultrasound image to one or more planar cuts of the segmented 3-D reference navigation image data in the memory 120. In one instance, the matching is achieved based on matching the segmented anatomy common in both image data sets, or sub-portions of that anatomy. Maximizing a number of anatomical structures segmented in 3-D reference navigation image data may provide a greatest opportunity to obtain common anatomy and unique planar cuts. However, the 3-D segmented anatomy may also be based on knowledge of segmentable anatomy within the real-time 2-D ultrasound plane(s).

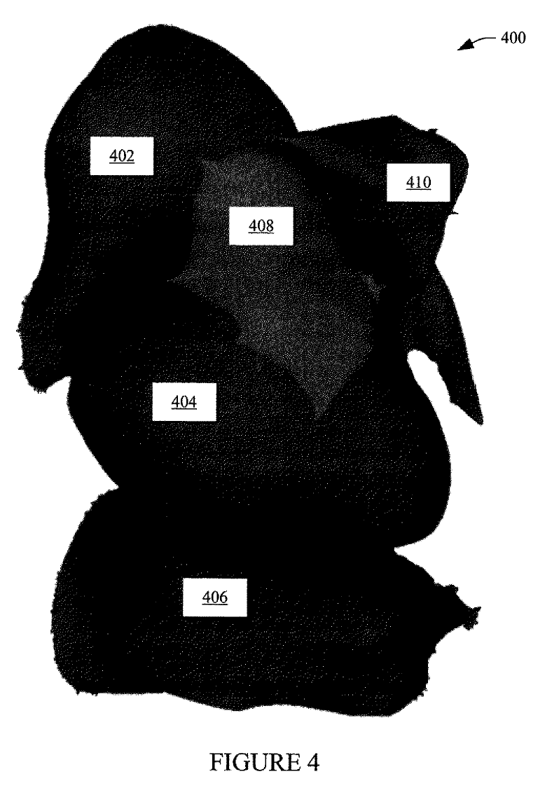

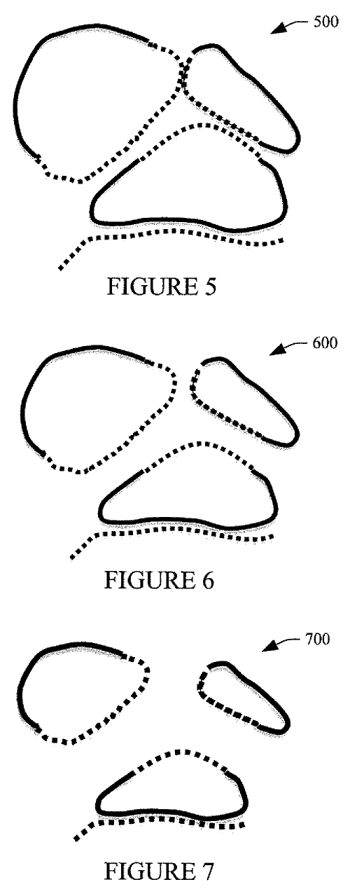

FIG. 3 depicts a real-time segmented 2-D ultrasound image 300 with contours of at least sub-portions of segmented anatomical structures 302, 304 and 306. FIG. 4 depicts an example of 3-D reference navigation image data 400 with a plurality of 3-D segmented structures 402, 404, 406, 408 and 410. FIGS. 5, 6 and 7 illustrate example planar cuts 500, 600 and 700 of the image data 400, which include contours of sub-portions of one or more of the segmented structures 402, 404, 406, 408 and 410. In these figures, first portions of the contours (shown as dotted lines) are also in FIG. 3, and second portions of the contours (shown as solid lines) are absent from FIG. 3. The real-time 2-D ultrasound image is matched to the more complete planar cuts 500, 600 and 700, derived from the preexisting 3D segmentation (e.g., a union of dotted and solid lines in FIGS. 5-7.

Matching the real-time 2-D ultrasound images to planar cuts can be accomplished, e.g., through template matching to a subset of planar cuts of the segmented 3-D reference navigation image data, where the subset is defined from knowledge of where the ultrasound probe is, generally, in relation to the scanned volume and what the orientation of the image plane(s) is. More specifically, sparse cuts of the 3-D volume, utilizing constraints imposed by the interaction of the ultrasound probe's geometry with the body, can be used in a first pass, to identify the general location of the probe, followed by locally more dense cuts to further localize the probe.

For example, maximum cone angle deviation (which would include yaw and pitch of the probe) from the “axis” of the tissue of interest and maximum rotation of the probe 102 about its axis (roll), relative to some reference direction (for example, the sagittal plane, that either bisects or bounds the anatomy of interest), while still intersecting the anatomy of interest in the image, are constraints imposed by the interaction of the probe geometry with the body. It is possible to leave out some of the real-time imaged anatomy, if one or more structures are suspected of being deformed, and therefore degrading the matching metric. This may be accomplished without loss of positioning accuracy and, may even produce an improvement.

The metric for “matching” a real-time ultrasound plane(s) with planar cuts of the previously segmented 3-D anatomical structures can be any metric that quantifies image similarity. For example, a normalized, zero-shift cross-correlation, or mutual information, may be used to measure the degree of match between a current plane(s) and one (or more) “test” planes extracted from the previously segmented 3D anatomical structures. Cross correlations or other matching metrics may optionally be done with bounded shifting of one plane relative to another, to account for slight misalignments due to modality differences, imperfect segmentations, and/or miss-registration.

The granularity of the calculation may be progressively increased to the full voxel density of the plane, to provide better discrimination between increasingly similar planes, as the set of selected planes becomes progressively more localized. This may require resampling of one plane to match the other, in the event that they are not collected at the same resolution, to allow mapping of equivalent voxel locations.

Segmented anatomy suspected of degrading the match, such as due to deformation, may be excluded from the matching. Such anatomy can be identified prior to the matching and excluded therefrom. In another instance, different anatomy is excluded during different matching iterations to determine what, if any, anatomy should be excluded from the matching. In yet another instance, an operator of the imaging system 100 identifies anatomy to exclude and/or include in the matching.

Internal checks for the consistency of positioning can be obtained: when using a single plane, one or more different subsets of the intersections can be matched to the planar cuts and the results compared to each other and/or to the full set; when data are collected from two planes (biplane probe) the second plane can be used to check the first, and/or any combination of subsets of 3-D surface intersections on either plane can be used to evaluate internal consistency.

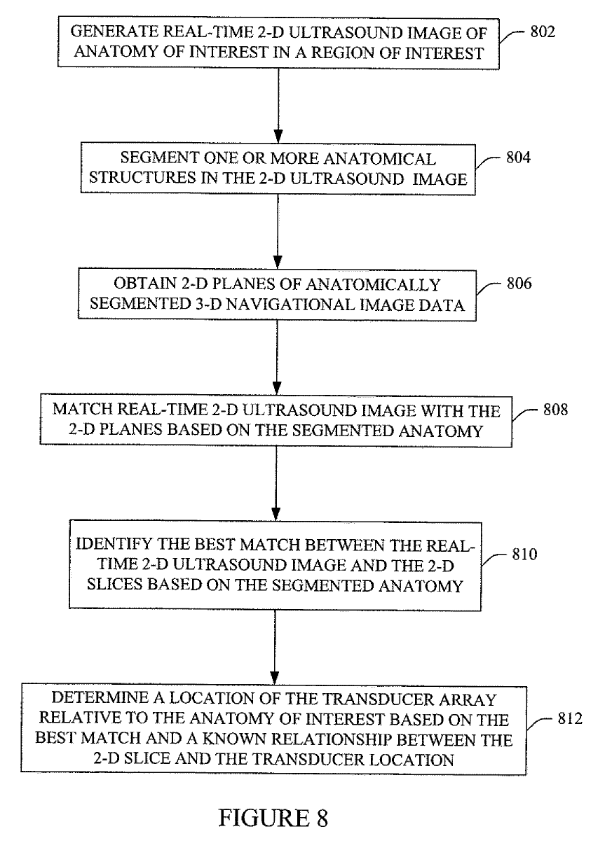

FIG. 8 illustrates an example method in accordance with an embodiment herein.

It is to be appreciated that the ordering of the above acts is not limiting. As such, other orderings are contemplated herein. In addition, one or more acts may be omitted and/or one or more additional acts may be included.

At 802, a real-time 2-D B-mode image of anatomy of interest in a region of interest is generated by the imaging system 100 with echoes received by the transducer elements 106.

At 804, one or more anatomical features are segmented from the real-time 2-D B-mode image, as described herein and/or otherwise.

At 806, 2-D slices of anatomically segmented 3-D navigation image data for the region of interest are obtained.

At 808, the real-time 2-D B-mode image is matched to the 2-D slices of the anatomically segmented 3-D navigation image data based on the segmented anatomy, as described herein and/or otherwise.

At 810, the 2-D slice of the anatomically segmented 3-D navigation image data that best matches the real-time 2-D B-mode image is identified, as described herein and/or otherwise.

At 812, a location and/or orientation of the transducer array 104 relative to the anatomy of interest is determined based on the best match and a known relationship between the 2-D slice and the transducer location, as described herein and/or otherwise.

At least a portion of the methods discussed herein may be implemented by way of computer readable instructions, encoded or embedded on computer readable storage medium (which excludes transitory medium), which, when executed by a computer processor(s), causes the processor(s) to carry out the described acts. Additionally or alternatively, at least one of the computer readable instructions is carried by a signal, carrier wave or other transitory medium.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A method, comprising:
obtaining a real-time 2-D B-mode image of anatomy of interest in a region of interest, wherein the real-time 2-D B-mode image is generated with ultrasound echoes sensed by transducer elements of a transducer array.
segmenting one or more anatomical features from the real-time 2-D B-mode image.
obtaining 2-D slices of anatomically segmented 3-D navigation image data for the same region of interest;
matching the real-time 2-D B-mode image to at least a sub-set of the 2-D slices based on the segmented anatomical features;
identifying a 2-D slice of the anatomically segmented 3-D navigation image data that matches the real-time 2-D B-mode image based on the matching; and
identifying at least one of a location and an orientation of the transducer array relative to the anatomy based on the match.

2. The method of claim 1, wherein the segmenting of the one or more anatomical features is based on at least one of a predetermined number of anatomical structures to segment, prior quantitative results, a data rate and a speed of motion.

3. The method of claim 2, further comprising:
dynamically determining the predetermined number based on a predetermined tradeoff between positioning accuracy and a speed of image update.

4. The method of claim 1, wherein the segmenting of the one or more anatomical features is based on knowledge of relative locations and boundaries of segmented anatomic structures in 3D reference image data.

5. The method of claim 1, wherein the segmented anatomy in the anatomically segmented 3-D navigation image data is based on segmentable anatomy within the real-time 2D ultrasound plane.

6. The method of claim 1, wherein the matching is based on template matching to the subset of planar cuts of the segmented 3-D image data.

7. The method of claim 1, wherein the subset is defined from knowledge of where the ultrasound probe is in relation to the scanned volume and an orientation of the image plane.

8. The method of claim 1, wherein the subset includes sparse cuts of the 3-D navigation image data utilizing constraints imposed by an interaction of a geometry of the ultrasound probe with an object being scanned to identify a general location of the probe followed by locally more dense cuts to further localize the probe.

9. The method of claim 1, wherein the matching is based on one or more of a normalized zero-shift cross-correlation, mutual information and cross-correlation.

10. The method of claim 1, further comprising:
excluding one or more of the segmented one or more anatomical features from the real-time 2-D B-mode image from the matching.

11. The method of claim 1, further comprising:
performing an internal check for a consistency of positioning.

12. The method of claim 11, wherein when using a single plane, one or more different subsets of intersections are matched to the planar cuts and results are compared to each other.

13. The method of claim 11, wherein when data are collected from two planes, one of the planes is used to check the first plane.

14. An apparatus, comprising:
a navigation processor configured to segment at least one anatomical organ of interest in a real-time 2-D ultrasound image and match the real-time 2-D ultrasound image to a 2-D slice of an anatomical segmented 3-D volume of image of interest based on common segmented anatomy in the real-time 2-D ultrasound image and the anatomical segmented 3-D volume of image of interest.

15. The apparatus of claim 14, wherein the navigation processor segments the at least one anatomical organ of interest in the real-time 2-D ultrasound image based on at least one of a predetermined number of anatomical structures to segment, prior quantitative results, a data rate and a speed of motion.

16. The apparatus of claim 14, wherein the navigation processor segments the at least one anatomical organ of interest in the real-time 2-D ultrasound image based on relative locations and boundaries of the 2-D slice of the anatomical segmented 3-D volume of image of interest.

17. The apparatus of claim 15, wherein the navigation processor excludes one or more of the segmented one or more anatomical features from the real-time 2-D B-mode image from the matching.

18. The apparatus of claim 15, wherein the navigation processor matches a single plane to two or more different 2-D slices.

19. The apparatus of claim 15, wherein the navigation processor matches two planes of a biplane transducer to a single 2-D slice.

20. A non-transitory computer readable medium encoded with computer executable instructions, which, when executed by a computer processor, causes the processor to:
segment one or more structure in a real-time 2-D ultrasound image;
match a contour of at least one of the segmented structures of the real-time 2-D ultrasound image with one or more contours of segmented anatomy in one or more planar cuts of 3-D image data including a planar cut corresponding to the real-time 2-D ultrasound image;
determine a location and an orientation of a transducer array using the matched plane(s) to obtain the real-time 2-D ultrasound image relative to the 3-D image data based on the match; and
display the 3-D image data with the real-time 2-D ultrasound image superimposed thereover at the determined location and orientation.
